(19) United States
(12) Patent Application Publication
(10) Pub. No.: US 2010/0180901 A1
(43) Pub. Date: Jul. 22, 2010

(54) CONFORMABLE DRAPE SYSTEM

(76) Inventor: Thomas H. Myers, Marietta, GA (US)

Correspondence Address: Brient Intellectual Property Law, LLC, 2300 Lakeview Parkway, Suite 700, Alpharetta, GA, 30009, US

(21) Appl. No.: 11/909,666

(22) PCT Filed: Mar. 27, 2006
(86) PCT No.: PCT/US06/11228

§ 371 Date: Sep. 25, 2007

Related U.S. Application Data
(60) Provisional application No. 60/667,951, filed on Mar. 25, 2005.

(51) Int. Cl. A61B 19/10; A61B 19/08

(52) U.S. Cl. 128/852; 128/849; 128/853

(57) ABSTRACT

A conformable drape system is disclosed that at least partially envelops an extremity or other portion of the body, for use during surgery or for the treatment of injuries. The drape may include a drape fabric, a flexible diaphragm positioned near a boundary of the sterile field, an inlet for receiving therapeutic fluid, and an outlet for discharging the fluid. The outlet may connect to a suction source to induce a pressure gradient throughout the drape, from the inlet toward the outlet. The suction may be used to withdraw excess air from inside the drape and thereby conform the drape more closely to the surface of the body. The fabric of the drape may include one or more features to enhance or facilitate the flow and dispersal of the fluid. The drape may be used in conjunction with a holder or cradle for positioning the extremity or other portion of the body and a tourniquet for limiting circulation. A method of use is also disclosed.

### BACKGROUND

1. Technical Field

The following disclosure relates generally to the protection and treatment of surgical sites and injuries and, more particularly, to a conformable drape system for the protection and preparation of surgical sites and injuries, especially on the extremities.

2. Description of Related Art

Conventional drape systems do not adequately contain surgical fluids. The escape of surgical fluids onto the floor of an operating room, for example, represents a contamination risk and a slipping hazard. Although some drape systems include portals and pouches for collecting fluids, surgical fluids such as blood and arthroscopic effluent inevitably escape and create a risk to both the patient and the staff. Conventional drape systems also do not provide a secure and predictable pathway for cleansing fluids and antiseptic solutions that may be applied to a surgical site.

Current drape systems also fail to provide a sufficiently sterile environment for the duration of a procedure or treatment, and often require several layers of material. Certain procedures and treatments require a relatively large sterile field, such as arthroscopic techniques that involve movement of a joint and positioning of an extremity. Moving an arm or leg away from the operating room table, for example, can bring the extremity into contact with a non-sterile environment. Conventional drape systems often fail to maintain the integrity of such a surgical field, especially when extremity movement is required.

Another problem with conventional drape systems is they are cumbersome and typically require application by two or more persons, including one in the surgical field using standard sterile technique. Preparing and draping a patient utilizing current drape systems often takes between ten and fifteen minutes, or longer. Although many disposable drapes are relatively inexpensive, they often consume significant time and expense because of the expertise required to apply the drape.

Many drape systems do not provide a complete barrier between the environment and the surgical field. Without a sufficient barrier, portions of the surgical field are at risk from dust, air currents, skin flora, and other impurities from the operating environment. Air currents induced by personnel entering and exiting the operating room suite, for example, have been shown to correlate with wound infection rates.

Current field dressings in use by emergency personnel and others to treat trauma and injuries also fail to provide a sufficient barrier between an open wound and the environment. Without a sufficient barrier, an open wound may be exposed to soil, sand, toxic substances, contaminants, debris, and any of a variety of other foreign matter found in the environment. The field dressings and bandages in use today do not create a sterile environment around an open wound. Also, many field dressings interfere with other treatments, such as the application of a brace or splint on an extremity. Current field dressings also fail to control bleeding at the injury site and, although they can be applied with some compression of the wound, most dressings do not provide a tourniquet effect on the entire extremity.

Current bandages and wound dressings also do not allow for the flow of fluids across and away from the site of an injury. Research has shown that lavage or irrigation of an open wound decreases infection rates. Unidirectional flow of fluids away from a surgical site or an injury is desirable, especially to remove contaminants.

Thus, there exists a need in the art for a drape system that provides improved fluid containment and a positional and mobile sterile field that is easy to apply and provides a more complete barrier to the environment. There is also a need in the art for a drape system the provides improved protection for trauma and injuries, such as open wounds. There is a related need in the art for a drape system that provides a secure and predictable pathway for cleansing irrigation fluids and antiseptic preparatory solutions applied to open wounds or surgical sites.

Certain illustrative and exemplary apparatuses, systems, and methods are described herein in connection with the following description and the accompanying drawing figures. The examples discussed represent only a few of the various ways of applying the principles supporting the material disclosed and, thus, the examples are intended to include equivalents. Other advantages and novel features may become apparent from the detailed description which follows, when considered in conjunction with the drawing figures.

### SUMMARY OF THE INVENTION

The following summary is not an extensive overview and is not intended to identify key or critical elements of the apparatuses, methods, systems, processes, and the like, or to delineate the scope of such elements. This Summary provides a conceptual introduction in a simplified form as a prelude to the more-detailed description that follows.

Certain illustrative example apparatuses, methods, systems, processes, and the like, are described herein in connection with the following description and the accompanying drawing figures. These examples represent but a few of the various ways in which the principles supporting the apparatuses, methods, systems, processes, and the like, may be employed and thus are intended to include equivalents. Other advantaged and novel features may become apparent from the detailed description that follows, when considered in conjunction with the drawing figures.

The above and other needs are met by the present invention which provides, in one embodiment, a system for draping a portion of the body to define a sterile field, comprising a drape including a fabric sized and shaped to cover a sterile field on a portion of the body, the sterile field extending from a proximal boundary to a distal boundary, the drape including a suction outlet, a connecting ring connected to the drape and including a flexible diaphragm and an injection port for receiving therapeutic fluid into the drape, and a suction outlet connected to the drape and to a suction system to withdraw air from inside the drape and otherwise draw the fabric toward the surface of the extremity and to induce a pressure gradient across the drape from the injection port toward the suction outlet. The diaphragm may be positioned near the proximal boundary and the suction outlet may be positioned near the distal boundary. The suction source may draw the therapeutic fluid through the drape toward the suction outlet.

The drape may include one or more directional features within the fabric to facilitate the flow of the therapeutic fluid within the drape. The drape may also include one or more dispersive features within the fabric to facilitate the dispersal of the therapeutic fluid across the surface of the extremity within the drape.

The connecting ring may include a circumferential channel in communication with the injection port, the channel including a series of inlets for dispersing the therapeutic fluid into the drape.

The drape may include one or more fenestrations positioned within the fabric. The fenestrations may be selectively openable from a closed position to an open position, with a bias toward the closed position. The connecting ring may include a rim for receiving the drape fabric.

The system may also include a holder for supporting the portion of the body, the holder including a cradle, the holder shaped to attach to the connecting ring. The holder may include a top that, together with the cradle at least partially surrounds the portion of the body, the top and cradle joined by a closure. The holder includes an interior groove for receiving a tourniquet. The tourniquet ma be selectively inflatable and may be placed around the portion of the body at a position proximal to the proximal boundary. The connecting ring may include a flange for connecting the ring to the holder.

The system may include a tourniquet that is selectively inflatable and may be placed around the portion of the body at a position proximal to the proximal boundary. For uses in which the portion of the body is an extremity, the distal boundary may be a distal end of the extremity, and the drape may be sized and shaped to at least partially envelop the extremity. The diaphragm may be sized and shaped to at least partially surround the extremity. The connecting ring may include a series of inlets in communication with the injection port and positioned to disperse the therapeutic fluid around the circumference of the extremity and into the drape. The holder may be sized and shaped to at least partially surround the extremity. The drape may carry a therapeutic agent disposed within the drape fabric.

In another aspect, the present invention may provide, in one embodiment, a system for draping an extremity of the body to define a sterile field, the system comprising a drape including a fabric sized and shaped to at least partially envelop an extremity and define a sterile field, the sterile field extending from a proximal boundary to a distal boundary, the drape including a suction outlet, a connecting ring connected to the drape and including a flexible diaphragm and an injection port for receiving therapeutic fluid into the drape, and a suction outlet connected to the drape and to a suction system to withdraw air from inside the drape and otherwise draw the fabric toward the surface of the extremity and to induce a pressure gradient across the drape from the injection port toward the suction outlet.

The diaphragm may be sized and shaped to at least partially surround the extremity. The connecting ring may include a series of inlets in communication with the injection port and positioned to disperse the therapeutic fluid around the circumference of the extremity and into the drape. The system may include a holder sized and shaped to at least partially surround the extremity.

The drape may include one or more directional features within the fabric to facilitate the flow of the therapeutic fluid within the drape. The drape may also include one or more dispersive features within the fabric to facilitate the dispersal of the therapeutic fluid across the surface of the extremity within the drape.

The connecting ring may include a circumferential channel in communication with the injection port, the channel including a series of inlets for dispersing the therapeutic fluid into the drape.

The drape may include one or more fenestrations positioned within the fabric. The fenestrations may be selectively openable from a closed position to an open position, with a bias toward the closed position. The drape may carry a therapeutic agent disposed within the drape fabric. The connecting ring may include a rim for receiving the drape fabric.

The system may also include a holder for supporting the portion of the body, the holder including a cradle, the holder shaped to attach to the connecting ring. The holder may include a top that, together with the cradle at least partially surrounds the portion of the body, the top and cradle joined by a closure. The holder includes an interior groove for receiving a tourniquet. The tourniquet ma be selectively inflatable and may be placed around the portion of the body at a position proximal to the proximal boundary. The connecting ring may include a flange for connecting the ring to the holder.

In another aspect, the present invention may provide a method for applying a surgical drape to a portion of the body. The method may begin with a preparation of the surface of the extremity with a cleansing or antiseptic solution. A tourniquet may be applied separately and positioned proximal to the proposed sterile field. The drape system of the present invention may be applied to a portion of the body, such as an extremity, as illustrated and described herein. The drape system may be used to exsanguinate or otherwise restrict circulation within an extremity or other part of the body.

These and other objects are accomplished by the present invention and will become apparent from the following detailed description of a preferred embodiment in conjunction with the accompanying drawings in which like numerals designate like elements.

### BRIEF DESCRIPTION OF THE DRAWING

The invention may be more readily understood by reference to the following description, taken with the accompanying drawing figures, in which:

FIG. 1 is an illustration of an apparatus, according to one embodiment of the present invention.

FIG. 2 is an exploded view of an apparatus, according to one embodiment of the present invention.

FIG. 3 is a side view of a drape, according to one embodiment of the present invention, applied to a leg.

FIG. 4 is a side view of a drape and a leg holder, according to one embodiment of the present invention, applied to a leg.

FIG. 5 is an illustration of a connecting ring, according to one embodiment of the present invention.

FIG. 6 is an exploded view of a leg holder and tourniquet, according to one embodiment of the present invention.

### DETAILED DESCRIPTION

### Introduction

Exemplary systems, methods, and apparatuses are now described with reference to the drawing figures, where like reference numerals are used to refer to like elements throughout the several views. In the following description, for purposes of explanation, numerous specific details are set forth in order to facilitate a thorough understanding of the systems, methods, apparatuses, and the like. It may be evident, however, that the exemplars described may be practiced without these specific details. In other instances, common structures and devices are shown in block diagram form in order to simplify the description.

Although the new systems, apparatuses, and methods will be more specifically described in the context of surgical procedures on extremities, the several aspects of the invention may also be advantageously applied for diagnostic or therapeutic purposes to other areas of the body.

To the extent that the term “includes” is employed in the detailed description or the list of exemplary inventive concepts, it is intended to be inclusive in a manner similar to the term “comprising” as that term is interpreted when employed as a transitional word in a claim. Further still, to the extent that the term “or” is employed in the list of exemplary inventive concepts (for example, A or B) it is intended to mean “A or B or both.” When the author intends to indicate “only A or B but not both,” the author will employ the phrase “A or B but not both.” Thus, use of the term “or” herein is the inclusive use, not the exclusive use. See Garner, A Dictionary Of Modern Legal Usage 624 (2d ed. 1995).

Many modifications and other embodiments may come to mind to one skilled in the art who has the benefit of the teachings presented in the description and drawings. It should be understood, therefore, that the invention is not be limited to the specific embodiments disclosed and that modifications and alternative embodiments are intended to be included within the scope of the disclosure and the claims. For example, it is contemplated that the present invention is not limited to the specific structures and shapes shown and described in the context of draping and treating a human extremity, such as the leg which is illustrated in the drawing figures. Other embodiments of the present invention may be developed and used without departing from the claimed subject matter. The present invention includes various other embodiments that are suitable, for example, for use on other portions of the human body or on animals. Although specific terms may be used herein, they are used in a generic and descriptive sense only and not for purposes of limitation.

As used herein, the term therapeutic fluid may include simple saline, antibiotic solutions, antibacterial washes, pharmaceutical liquids, or other such fluids that provide cleansing, prevent infection, promote healing, reduce pain, or are otherwise therapeutic.

The various embodiments of the present invention may be described herein in the context of a human extremity, such as the arm or leg. In general, an extremity has a proximal portion near the torso and an opposing distal portion. The relative motion of elements or the flow of therapeutic fluids, for example, may be described as proceeding proximally (toward the torso) or distally (away from the torso).

### Conformable Drape

In one embodiment, the present invention may include a sterile enclosure or drape that at least partially envelops an extremity or other portion of the body. The drape may define a sterile field, from a proximal boundary to a distal boundary, which may be at the distal end of an extremity. The drape may include a flexible diaphragm positioned near the proximal boundary of the sterile field. The drape may include an inlet for receiving a therapeutic fluid and an outlet for discharging the fluid. The outlet may connect to a source of suction, thereby creating a pressure gradient throughout the drape, from the inlet toward the outlet. The suction may be used to withdraw excess air from inside the drape and thereby conform the drape more closely to the surface of the body. The fabric of the drape may include one or more features to enhance or facilitate the flow and dispersal of the fluid as it passes over the extremity. The drape, in one embodiment, may be used in conjunction with a cradle for an extremity or other portion of the body. The cradle may include a tourniquet.

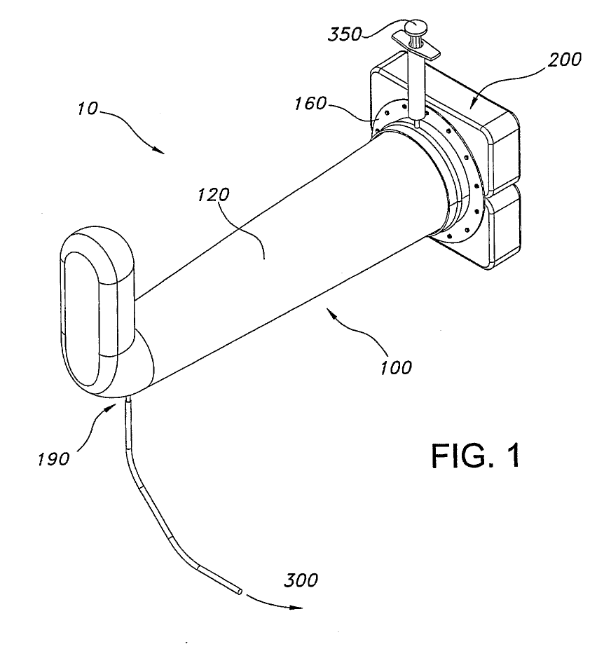

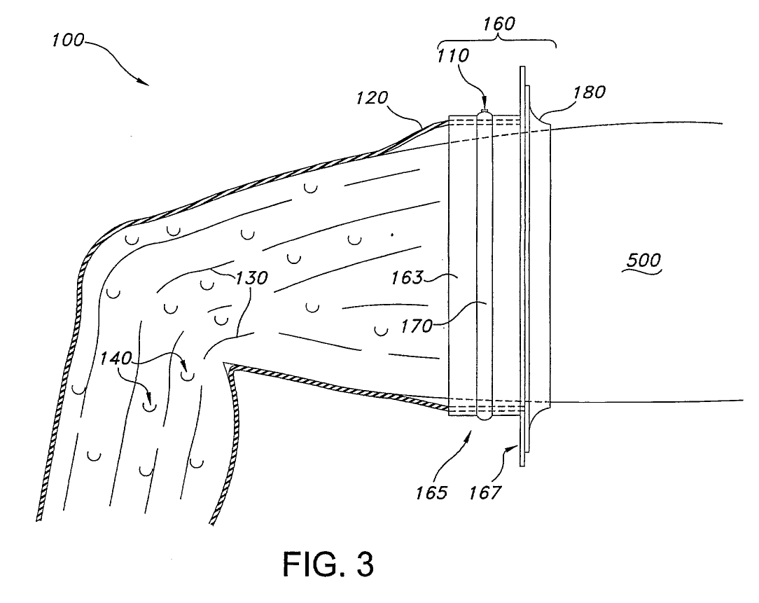

As shown in FIG. 1, the apparatus 10 of the present invention, in one embodiment, may include a sterile enclosure or drape 100 and a cradle or leg holder 200. The drape 100 may be sized and shaped to at least partially envelop an extremity or other portion of the body. The drape 100 may include a flexible diaphragm 180 (shown in FIG. 3) which may be placed at the proximal boundary of the sterile field. The drape 100 may also include a drape fabric 120, a connecting ring 160, and an inlet or injection port 110. The injection port 110 may receive any of a variety of therapeutic fluids, using an injection system or syringe 350, for example. The drape 100 may include an outlet 190 for discharging the fluids. The outlet 190 may provide suction, through the force of gravity or through a connection to a suction system 300, thereby creating a pressure gradient throughout the drape 100, from the inlet port 110 toward the outlet 190. The suction may be used to withdraw excess air from inside the drape 100 and thereby conform the drape 100 more closely to the surface of the body. The drape fabric 120, as described in more detail below, may include one or more features to enhance or facilitate the flow and dispersal of the fluid as it passes over the extremity. The drape 100, in one embodiment, may or may not be used in conjunction with a cradle for an extremity such as a leg holder 200, as shown, or a cradle shaped to support any other portion of the body. The cradle or leg holder 200 may include a tourniquet 280.

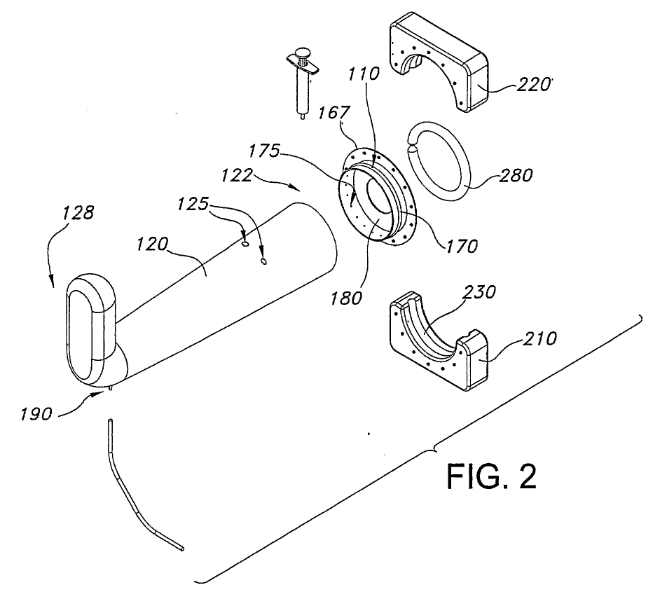

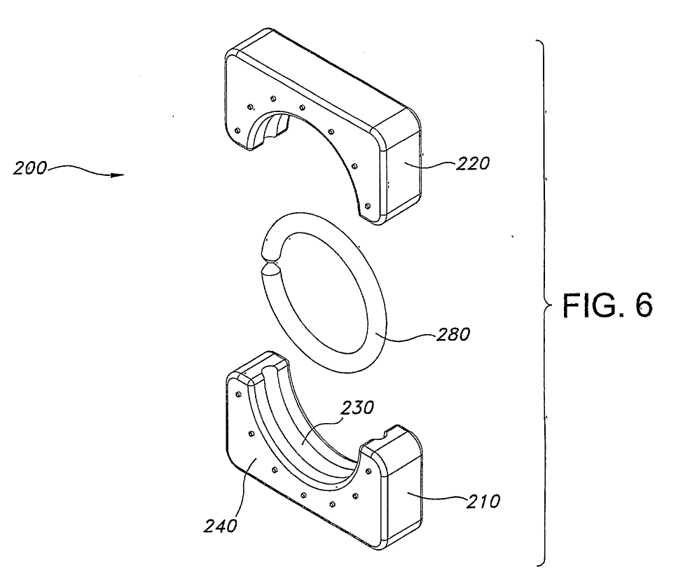

FIG. 2 is an exploded, isometric view of the embodiment illustrated in FIG. 1. The leg holder 200, as shown, may include a cradle 210 to support a leg or other extremity, and it may or may not include a top 220. The leg holder 200 may also include a tourniquet 280. In one embodiment, the tourniquet 280 may be selectively inflatable, and the leg holder 200 may include one or more ports for connecting an external inflation source for the tourniquet 280. The leg holder 200 may include an interior grove or channel 230 that is sized and shaped to receive the tourniquet 280. The leg holder 200, in one embodiment, is also shown in FIG. 6. Although the leg holder 200 may be referred to herein in the context of supporting a leg, the holder 200 may be sized and shaped to support any extremity or other part of the body. In general, the leg holder 200 may have a diameter sized to fit the largest leg expected to be encountered in use, such that the leg holder 200 may be used with legs of any size. In the embodiment that includes a tourniquet 280, the holder 200 may be sized to fit and positioned to facilitate application of the tourniquet 280 at a preferred location on the body.

The drape may include one or more fenestrations 125, as shown in FIG. 2, to facilitate the insertion of surgical instruments. The fenestrations 125 may be sealed initially, and may be re-sealable. The fenestrations 125 may be selectively openable, from a closed position to an open position, with a bias toward said closed position. Insertion may also be accomplished through simple incisions made in the drape fabric 120 at locations desirable for the procedure being performed. Also, the drape 100 may be used in conjunction with one or more auxiliary drapes or drape systems, as desired.

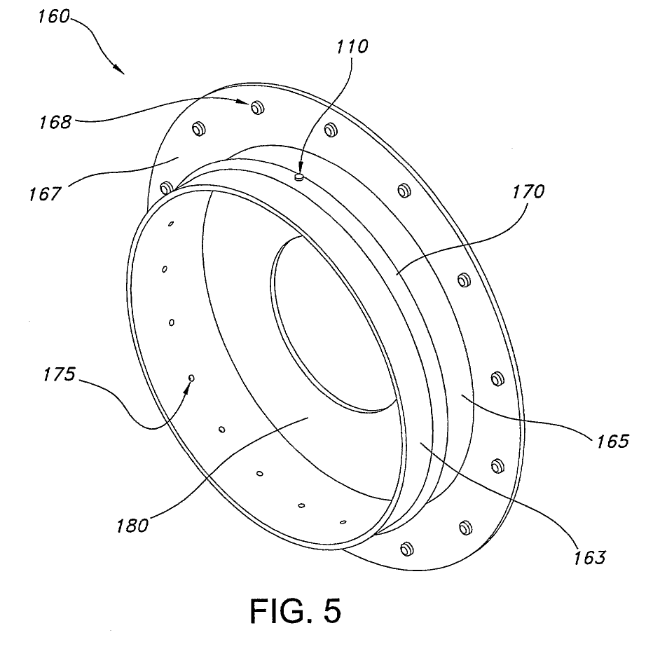

The connecting ring 160, as shown in FIG. 2 and FIG. 5, may include a flexible diaphragm 180 that is sized and shaped to fit around an extremity or other body part. In use, the ring 160 may be placed on the patient by passing the extremity through a central opening in the diaphragm 180 and moving the ring 160 into a desired position. The connecting ring 160 may also include an injection port 110 that, in one embodiment, may be connected to a circumferential channel 170 or passageway around the circumference of the ring 160. The channel 170 may include one or more inlets 175 on the interior surface of the connecting ring 160. In one embodiment, the inlets 175 may be distributed in a series around the circumference of the ring 160 in order to evenly disperse a therapeutic fluid around the extremity. Fluids may be injected through the injection port 110 and, through the channel 170 and inlets 175, may be distributed evenly across the extremity or body part to be treated. The series of inlets 175 is also shown in FIG. 5.

The drape fabric 120, as shown in FIG. 2, may have a distal end 128 and a proximal end 122. In one embodiment, the proximal end 122 of the drape fabric 120 may be attached to the connecting ring 160. The drape fabric 120 may also include one or more windows or fenestrations 125. The fenestrations 125 may be used as surgical access ports, for the insertion of surgical instruments, or to otherwise gain access to a wound or surgical site on the body. The drape fabric 120 may also include a suction outlet 190 that may be positioned near the distal end 128 or at any other location that may facilitate proper suction.

FIG. 3 is a side view of an extremity 500 (a leg) that has been inserted into a drape 100. The connecting ring 160, as shown, has been placed around the thigh, just above the knee. The drape fabric 120 as shown surrounds the knee area and extends down the leg distally. The connecting ring 160, as shown, may include a ring body 165 and a flange 167. The flange 167 may be sized and shaped to be connected to a leg holder 200. The body 165 of the connecting ring 160 may include a rim 163, which may be used to receive an edge of the drape fabric 120, as shown. The drape fabric 120 may be adhered to the rim 163 of the connecting ring 160 using any of a variety of attachment means. The side view of the connecting ring 160 in FIG. 3 illustrates the circumferential channel 170 which may be centrally located on the body 165 of the connecting ring 160. The injection port 110, as shown, at the top, may be used to introduce therapeutic fluids directly into the circumferential channel 170. Also shown in FIG. 3 is the flexible diaphragm 180 which surrounds the upper leg or thigh. The diaphragm 180, in one embodiment, may be used to create a seal around the extremity 500, thereby creating a barrier and a proximal boundary for the sterile field. In this aspect, the diaphragm 180 may serve as the uppermost or proximal barrier, defining the proximal extent of the sterile field. The embodiment shown in FIG. 3 does not include a leg holder 200. In one embodiment, the connecting ring 160 itself may act as a leg holder, providing support to a leg or other extremity upon which the drape 100 may be applied. In this aspect, the connecting ring 160 may provide a stable connecting point for the drape 100 in situations that do not require anchoring of the extremity, for example, to an operating room table. Also, the connecting ring 160 may be used without a leg holder 200 in situations where more mobility of the extremity is desired.

The series of inlets 175, shown in FIG. 5, may be used to evenly distribute a therapeutic fluid around the circumference of the extremity 500. Referring to FIG. 3, the fluid entering the injection port 110 may enter the channel 170 and then pass through the series of inlets 175 (inside the ring 160) which are directed generally toward the extremity 500. The inlets 175 may include a nozzle or other apparatus to control the disbursal of the fluid. In this aspect, the channel 170 and the series of inlets 175 may be configured to disperse a fluid only when a certain baseline pressure has been reached. Following the path of the fluid, in FIG. 3, as it is distributed around the surface of the extremity 500, it may proceed distally along the suction gradient, or generally downward with gravity. It may be noted that the general diameter of the leg 500 is somewhat smaller than the diameter of the body 165 of the connecting ring 160. The connecting ring 160 may be sized and shaped to accommodate the largest extremity that may be encountered. In this aspect, the drape 100 of the present invention may fit any size extremity. Because the ring 160 is generally larger than the extremity 500, there will be a gap or tent near the ring 160, between the drape fabric 120 and the surface of the extremity 500 itself. It is through this gap or tent where the fluid or solution may start to flow out of the series of inlets 175 and onto the surface of the extremity 500. The tent portion beneath the drape fabric 120 is illustrated in FIG. 3. Moving distally, or down the leg, the drape fabric 120 may conform much more closely to the surface of the extremity 500, as shown. In one embodiment, suction may be applied to the drape 100 to urge the drape fabric 120 closer to the surface of the extremity 500.

### Drape Fabric, Pressure Gradient, and Fluid Flow

In one embodiment, the drape fabric 120 may be made of a material that is flexible and resistant to tearing, to facilitate safe application of the drape 100 without damaging the fabric 120. The material may be translucent or clear, or nearly transparent, to promote visibility of the extremity. The drape fabric 120, in one embodiment, may include multiple layers of material. For example, one layer may include depots filled with therapeutic fluids or it may be impregnated with antiseptic solution, for example, that will elute or otherwise disperse from the fabric 120 across all or most of the surface area of the extremity. Another layer may be coated with a sponge-like texture or material that can absorb and then disperse therapeutic fluids.

In one embodiment, the drape fabric 120 may have an inner texture or topography. A smooth fabric might adhere too closely to the skin of the extremity and may prevent the flow of therapeutic fluids, which may be desirable. During surgical procedures or for treatment of an open wound, the drape fabric 120 of the present invention may provide a pathway for the introduction and collection of therapeutic fluids across and through a sterile field. In order to promote the flow of therapeutic fluid and the dispersal of the fluid around the extremity 500, the drape fabric 120 may include directional features 130 and/or dispersive features 140.

The directional features 130 may include channels or grooves in the interior surface of the drape fabric 120 that are sized and shaped to direct or channel a fluid so that it will move efficiently across the surface of the extremity 500. Because the drape 100 may be used in conjunction with a suction outlet 190 and the operating room suction source 300, there may be a pressure gradient induced across the sterile field. In this aspect, the pressure gradient will tend to promote fluid flow distally from the injection port 110, across the surface of the extremity 500, toward the distal end 128 of the drape 100, and out through the suction outlet 190. The directional features 130 may include any of a variety of shapes designed to promote flow. In one embodiment the directional features 130 may include linear channels or grooves extending lengthwise along the surface of the extremity 500. In another embodiment the directional features 130 may include spiral grooves or rifling, which may be sized and shaped to direct the therapeutic fluid in a spiral pattern around the surface of the extremity 500 along its path toward the suction outlet 190.

The one or more dispersive features 140 may be sized and shaped to disperse or disseminate a therapeutic fluid across the surface of the extremity 500. The dispersive features 140 may include a number of recesses or cavities in the drape fabric 120 which may have the appearance of bubbles or pockets. As the fluid flows across the surface of the extremity 500, the dispersive features 140 may be used to temporarily trap or slow the progress of the fluid so that it reaches areas of the surface of the extremity 500 where the fluid may provide a therapeutic benefit. In this aspect, the dispersive features 140 may provide a means for placing therapeutic fluids across wide areas of a sterile field.

The drape fabric 120 may include one or more kinds of directional features 130 and/or one or more types of dispersive features 140, depending upon the application, the desired flow pattern, the desired dispersal rate, the procedure being performed, and the type of wound or other condition to be treated.

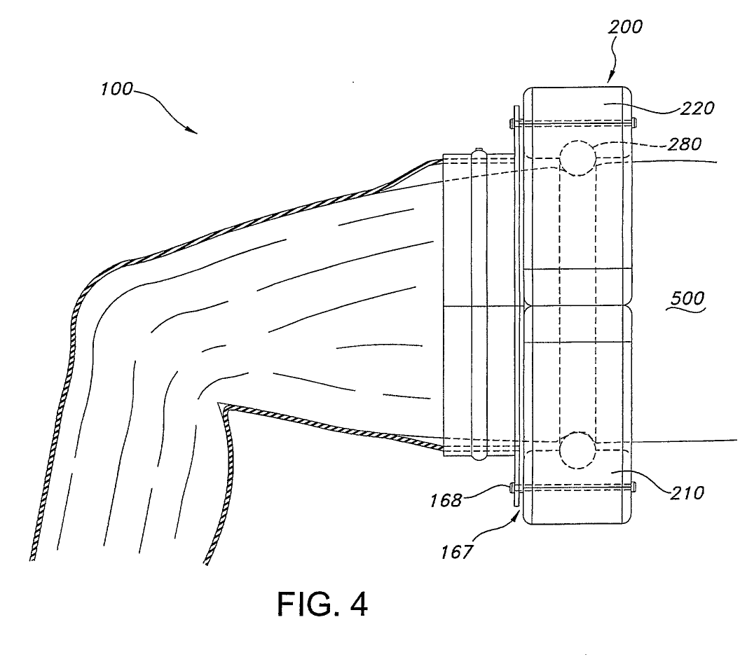

FIG. 4 is an illustration of the drape 100 attached to a leg holder 200. As shown, in one embodiment, the flange 167 of the connecting ring 160 may be attached to the leg holder 200 using one or more fasteners 168. The illustration of the leg holder 200 includes a cradle 210 generally positioned underneath the extremity 500 and a top portion 220. The cradle 210 and top 220 may be joined using any of a variety of connecting means including a hinge or other type of closure. Inside the leg holder 200 may be a tourniquet 280 positioned around the extremity 500 to halt the flow of blood. In one embodiment, the tourniquet 280 is selectively inflatable using a source of inflation and deflation located outside the leg holder 200. There may be one or more portals through which an exterior source of inflation may be connected to the tourniquet 280 while the leg holder 200 is in place around the extremity 500 of the patient.

In one embodiment, the drape 100 of the present invention may include one or more auxiliary drapes or draping systems. An auxiliary drape may be used to provide improved fluid containment. For example, an auxiliary drape with an adhesive perimeter may be used to contain fluid that might exit the wounds when an arthroscope is withdrawn from the knee or from a cannula that remains within the knee. The auxiliary drape may also include small diaphragms or one-way passages that allow insertion and extraction of instruments into and out of the wound or surgical port. The diaphragms or passages may contain fluid by immediately sealing around the instrument and closing when the instrument is withdrawn. Auxiliary drapes may be attached to a separate suction source or simply contain the fluids in a pouch. They also may be attached to the main drape with connections to the inside sterile field. These auxiliary drapes may also be attached to other devices, such as a pulsed lavage system.

### Illustrative Use of the Drape System

The following method steps are described in the context of applying a surgical drape to a leg, although it could be applied to another extremity or portion of the body. In one embodiment, the present invention may include a method of applying or installing a conformable surgical drape of the present invention. The method may begin with a preparation of the surface of the extremity 500 with a cleansing or antiseptic solution. The tourniquet 280 may be applied separately, around the proximal thigh, for example, and positioned proximal to the proposed sterile field. The tourniquet 280 may be oriented to fit within the cradle 210 of the leg holder 200. With the tourniquet 280 in position, inflated or not, the leg may be placed in the cradle 210 of the leg holder 200 by placing the thigh on top of the cradle 210 such that the tourniquet 280 rests in the channel 230. Alternatively, the tourniquet 280 may be an inflatable bladder that is attached to the leg holder 200 and clamped around the thigh when the leg holder 200 is closed and tightened.

Next, the foot may be placed through the diaphragm 180 and into the drape fabric 120. By sliding the diaphragm 180 proximally, up the leg, the drape fabric may be applied around the lower leg like a stocking. The diaphragm 180 may be placed on the thigh near the tourniquet such that the ring flange 167 is near the distal face 240 of the leg holder 200. At this point, the top 220, if provided, may be connected to the cradle 210 to complete or otherwise close the leg holder 200. The connecting ring 160 may then be attached to the leg holder 200 using one or more fasteners 168. At this stage, the apparatus 10 of the present invention may appear similar to the illustration in FIG. 4.

In preparation for a surgical procedure, for example, it may be advisable to exsanguinate the leg or other extremity. One method of exsanguination is to apply a rubber wrap, starting at the foot, and wrapping the leg very tightly in the proximal direction to force blood out of the limb. Once the exsanguination wrap reaches the thigh, the tourniquet 280 may be inflated or otherwise applied across the thigh. After the tourniquet 280 is applied, the exsanguination wrap may be unwound and removed from the leg.

In one embodiment, instead of exsanguinating the limb using the wrap method, the suction system 300 may be used to apply suction to the drape 100. As the air is removed from the drape and it shrinks around the surface of the extremity 500, the blood may be forced out of the limb, proximally, thus having the same effect as the exsanguination wrap method. Once the suction reaches a desirable level and it produces enough exsanguination in the leg, the tourniquet 280 may be inflated or otherwise applied.

After the tourniquet is applied and the exsanguination has been completed, the suction system 300 may be used to withdraw air from around the extremity 500 and otherwise shrink the drape 100 such that the drape fabric 120 shrinks toward and engages the exterior surface of the extremity 500. A base level of suction may be continuously applied to the drape 100 to maintain a pressure gradient distally. The pressure gradient may be used to facilitate the introduction of a therapeutic fluid, which may be introduced at any time during a procedure or treatment. Using the injection system 350, such as the simple syringe illustrated in FIG. 1, a therapeutic fluid may be applied through the injection port 110 into the drape 100. The injection system 350 may be a pulsed lavage system. The pressure gradient will draw the fluid down the leg, distally, and accomplish a wash of the entire sterile field. The suction outlet 190 will act as a drain for the suction system 300 to carry away the fluid. This cycle may be applied during a surgical procedure or to flush an open wound positioned inside the drape 100 or may be otherwise used to sterilize or facilitate the sterilization of the surgical field.

In one embodiment the drape 100 of the present invention may be used without the leg holder 200. As shown in FIG. 3, the diaphragm 180 provides a proximal border on the sterile field while the pressure of the drape under suction may provide sufficient exsanguination and tourniquet effect to allow a surgical procedure to be accomplished without a tourniquet. In one embodiment, the lower portion of the connecting ring 160 may include a cradle for the thigh.

### Field Drape

In another embodiment, the apparatus 10 of the present invention may be applied in the field as a dressing for an open wound or other injury. The drape 100 may include the drape fabric 120 and a ring 160 modified for use in the field. In one embodiment, the ring 160 may include all the elements described above except for the flange 167, which may not be necessary in the field. The drape 100 may be applied to any extremity in order to protect the wound from debris and other foreign matter. Suction may be applied using a portable suction system 300 attached to the suction outlet 190 in order to shrink the drape fabric 120 around the extremity 500 as described. Saline or another therapeutic fluid may be injected through the injection port 110 and the ring 160 and allowed to travel across the extremity 500 and through the suction outlet 190. In use, the drape 100 may provide a tourniquet effect to help slow the bleeding in an open wound. It should be noted, however, that any major arterial bleeder should be clamped or otherwise staunched before applying the drape 100 of the present embodiment. The drape 100 may also provide a certain amount of stabilization for the extremity, such as might be needed when there is loss of soft tissue or a fracture or other serious injury to the extremity. In this aspect, the drape 100 may act as a second skin and a temporary sterile field for the application of sterile therapeutic fluids to wash the affected extremity. The portable suction system may include a simple syringe or other kind of manual suction device, or it may alternatively include a powered unit configured to provide suction in the field. Because the drape fabric 120 may be drawn close to the surface of the extremity 500, any sort of splint or cast or brace may be applied over the drape 100, without interference, to further stabilize the extremity so the patient may be transported. In this aspect, the drape 100 does not restrict or limit the ability to splint the extremity.

The field drape 100 may also include one or more access ports or fenestrations 125 that are initially sealed shut, to maintain the partial vacuum around the extremity, but can be easily opened or breached to insert any number of devices to irrigate, pack, debride, or otherwise manipulate the wound. Such devices may include a pulsed lavage, syringe irrigation, bandages or wound packs, clamps, or other devices to introduce coagulants, other pharmaceuticals, or therapeutic fluids to the wound site.

### CONCLUSION

Although the systems, apparatuses, and methods herein have been illustrated by describing examples, and while the examples have been described in considerable detail, the description is not exhaustive. It is not possible, of course, to describe every conceivable combination of components or methodologies for purposes of describing the systems, apparatuses, and methods for treating a fracture site. One of ordinary skill in the art may recognize that further combinations and permutations are possible. Accordingly, this application is intended to embrace alterations, modifications, and variations that fall within the scope of the appended claims. Furthermore, the preceding description is not meant to limit the scope of the invention. Rather, the scope of the invention is to be determined only by the appended list of exemplary inventive concepts and their equivalents.

1. In a system for draping a portion of the body to define a sterile field, the system characterized by:
a drape (100) including a fabric (120) sized and shaped to cover a sterile field on a portion of the body, said sterile field extending from a proximal boundary to a distal boundary, said drape including a suction outlet (190);
a connecting ring (160) connected to said drape (100) and including a flexible diaphragm (180) and an injection port (110) for receiving therapeutic fluid into said drape (100); and
a suction outlet (190) connected to said drape (100) and to a suction system (300) to withdraw air from inside said drape and otherwise draw said fabric (120) toward the surface of said extremity and to induce a pressure gradient across said drape from said injection port (110) toward said suction outlet (190).

2. The system of claim 1, further characterized in that said diaphragm (180) is positioned near said proximal boundary and said suction outlet (190) is positioned near said distal boundary.

3. The system of claim 1, further characterized in that said suction source (300) draws said therapeutic fluid through said drape (100) toward said suction outlet (190).

4. The system of claim 1, further characterized in that said drape (100) includes one or more directional features (130) within said fabric (120) to facilitate the flow of said therapeutic fluid within said drape (100).

5. The system of claim 1, further characterized in that said drape (100) includes one or more dispersive features (140) within said fabric (120) to facilitate the dispersal of said therapeutic fluid across said surface of said extremity within said drape (100).

6. The system of claim 1, further characterized in that said connecting ring (160) includes a circumferential channel (170) in communication with said injection port (110), said channel (170) including a series of inlets (175) for dispersing said therapeutic fluid into said drape (100).

7. The system of claim 1, further characterized in that said drape (100) includes one or more fenestrations (125) positioned within said fabric (120).

8. The system of claim 1, further characterized in that said drape (100) includes one or more fenestrations (125) positioned within said fabric (120), wherein said one or more fenestrations (125) is selectively openable from a closed position to an open position, with a bias toward said closed position.

9. The system of claim 1, further characterized in that said connecting ring (160) includes a rim (163) for receiving said drape fabric (120).

10. The system of claim 1, further characterized by a holder (200) for supporting said portion of the body, said holder including a cradle (210), said holder shaped to attach to said connecting ring (160).

11. The system of claim 10, further characterized in that said holder (200) includes a top (220) that, together with said cradle (210) at least partially surrounds said portion of the body, said top and cradle joined by a closure.

12. The system of claim 10, further characterized in that said holder (200) includes an interior groove (230) for receiving a tourniquet (280).

13. The system of claim 10, further characterized in that said holder (200) includes an interior groove (230) for receiving a tourniquet (280), wherein said tourniquet (280) is selectively inflatable and placed around said portion of the body at a position proximal to said proximal boundary.

14. The system of claim 10, further characterized in that said connecting ring (160) includes a flange (167) for connecting said ring (160) to said holder (200).

15. The system of claim 1, further characterized by a tourniquet (280) that is selectively inflatable and placed around said portion of the body at a position proximal to said proximal boundary.

16. The system of claim 1, further characterized in that said portion of the body is an extremity, said distal boundary is a distal end of said extremity, and said drape (100) is sized and shaped to at least partially envelop said extremity.

17. The system of claim 16, further characterized in that said diaphragm (180) is sized and shaped to at least partially surround said extremity.

18. The system of claim 16, further characterized in that said connecting ring (160) includes a series of inlets (175) in communication with said injection port (110) and positioned to disperse said therapeutic fluid around the circumference of said extremity and into said drape (100).

19. The system of claim 16, further characterized by a holder (200) sized and shaped to at least partially surround said extremity.

20. The system of claim 1, further characterized in that said drape (100) carries a therapeutic agent disposed within said drape fabric (120).

21. In a system for draping an extremity of the body to define a sterile field, the system characterized by:
a drape (100) including a fabric (120) sized and shaped to at least partially envelop an extremity and define a sterile field, said sterile field extending from a proximal boundary to a distal boundary, said drape including a suction outlet (190);
a connecting ring (160) connected to said drape (100) and including a flexible diaphragm (180) and an injection port (110) for receiving therapeutic fluid into said drape (100); and
a suction outlet (190) connected to said drape (100) and to a suction system (300) to withdraw air from inside said drape and otherwise draw said fabric (120) toward the surface of said extremity and to induce a pressure gradient across said drape from said injection port (110) toward said suction outlet (190).

22. The system of claim 21, further characterized in that said diaphragm (180) is sized and shaped to at least partially surround said extremity.

23. The system of claim 21, further characterized in that said connecting ring (160) includes a series of inlets (175) in communication with said injection port (110) and positioned to disperse said therapeutic fluid around the circumference of said extremity and into said drape (100).

24. The system of claim 21, further characterized by a holder (200) sized and shaped to at least partially surround said extremity.

25. The system of claim 21, further characterized in that said diaphragm (180) is positioned near said proximal boundary and said suction outlet (190) is positioned near said distal boundary.

26. The system of claim 21, further characterized in that said suction source (300) draws said therapeutic fluid through said drape (100) toward said suction outlet (190).

27. The system of claim 21, further characterized in that said drape (100) includes one or more directional features (130) within said fabric (120) to facilitate the flow of said therapeutic fluid within said drape (100).

28. The system of claim 21, further characterized in that said drape (100) includes one or more dispersive features (140) within said fabric (120) to facilitate the dispersal of said therapeutic fluid across said surface of said extremity within said drape (100).

29. The system of claim 21, further characterized in that said drape (100) carries a therapeutic agent disposed within said drape fabric (120).

30. The system of claim 21, further characterized in that said connecting ring (160) includes a circumferential channel (170) connected to said injection port (110), said channel (170) including a series of inlets (175) for dispersing said therapeutic fluid into said drape (100).

31. The system of claim 21, further characterized in that said drape (100) includes one or more fenestrations (125) positioned within said fabric (120).

32. The system of claim 21, further characterized in that said drape (100) includes one or more fenestrations (125) positioned within said fabric (120), wherein said one or more fenestrations (125) is selectively openable from a closed position to an open position, with a bias toward said closed position.

33. The system of claim 21, further characterized in that said connecting ring (160) includes a rim (163) for receiving said drape fabric (120).

34. The system of claim 21, further characterized by a holder (200) for supporting said portion of the body, said holder including a cradle (210), said holder shaped to attach to said connecting ring (160).

35. The system of claim 34, further characterized in that said holder (200) includes a top (220) that, together with said cradle (210) at least partially surrounds said portion of the body, said top and cradle joined by a closure.

36. The system of claim 34, further characterized in that said holder (200) includes an interior groove (230) for receiving a tourniquet (280).

37. The system of claim 34, further characterized in that said holder (200) includes an interior groove (230) for receiving a tourniquet (280), wherein said tourniquet (280) is selectively inflatable and placed around said portion of the body at a position proximal to said proximal boundary.

38. The system of claim 34, further characterized in that said connecting ring (160) includes a flange (167) for connecting said ring (160) to said holder (200).

39. The system of claim 21, further characterized by a tourniquet (280) that is selectively inflatable and placed around said portion of the body at a position proximal to said proximal boundary.
